(19) United States
(12) Patent Application Publication
(10) Pub. No.: US 2024/0198179 A1
(43) Pub. Date: Jun. 20, 2024

(54) CYCLING SPORT TRAINING SUGGESTION SYSTEM

(71) Applicant: UNIWILL TECHNOLOGY INC., TAIPEI CITY (TW)

(72) Inventors: Chien-Yuan CHEN, TAIPEI CITY (TW); Chun-Cheng CHEN, TAIPEI CITY (TW); Shao-Hong YANG, TAIPEI CITY (TW); Jen-Sheng TSAI, TAIPEI CITY (TW)

(21) Appl. No.: 18/126,389

(22) Filed: Mar. 25, 2023

(30) Foreign Application Priority Data
Dec. 19, 2022 (TW) 111148750

(51) Int. Cl. A63B 24/00; A63B 69/16; A63B 71/06

(52) U.S. Cl. CPC A63B 24/0075; A63B 69/16; A63B 71/0622; A63B 2024/0068; A63B 2220/30; A63B 2230/06

(57) ABSTRACT

A cycling sport training suggestion system (10) includes a sport real-time analysis module (102), a cyclist information module (104), a route feature module (106) and a sport role data base (108). The cyclist information module (104) transmits a cyclist real-time sense information (110) to the sport real-time analysis module (102). The route feature module (106) transmits a route feature information (112) to the sport real-time analysis module (102). The sport role data base (108) transmits a cyclist historical sport information (114) and a perfect cyclist sport information (116) to the sport real-time analysis module (102). The sport real-time analysis module (102) integrates and analyzes the cyclist real-time sense information (110), the route feature information (112), the cyclist historical sport information (114) and the perfect cyclist sport information (116) to generate a cycling sport training suggestion signal (118).

### BACKGROUND OF THE DISCLOSURE

### Technical Field

The present disclosure relates to a training suggestion system, and especially relates to a cycling sport training suggestion system.

### Description of Related Art

Cycling may promote health and increase the enjoyment of life, so that many people are keen on cycling at present.

In order to strengthen the skills of cycling, the cyclist may wear some apparatuses to sense the cyclist's information such as the heart rate and so on, so as to understand the cyclist's physical condition.

However, currently there is a lack of a system that may simultaneously integrate multiple information to provide suggestions for the cyclist riding a specific route, so that the cycling efficiency of the cyclist may not be effectively improved, which is a pity.

### SUMMARY OF THE DISCLOSURE

In order to solve the above-mentioned problems, an object of the present disclosure is to provide a cycling sport training suggestion system.

In order to achieve the object of the present disclosure mentioned above, the cycling sport training suggestion system of the present disclosure includes a sport real-time analysis module, a cyclist information module, a route feature module, and a sport role data base. The cyclist information module is electrically connected to the sport real-time analysis module. The route feature module is electrically connected to the sport real-time analysis module. The sport role data base is electrically connected to the sport real-time analysis module, the cyclist information module, and the route feature module. Moreover, the cyclist information module is configured to transmit a cyclist real-time sense information to the sport real-time analysis module. The route feature module is configured to transmit a route feature information to the sport real-time analysis module. The sport role data base is configured to transmit a cyclist historical sport information and a perfect cyclist sport information to the sport real-time analysis module. After the sport real-time analysis module receives the cyclist real-time sense information, the route feature information, the cyclist historical sport information and the perfect cyclist sport information, the sport real-time analysis module is configured to integrate and analyze the cyclist real-time sense information, the route feature information, the cyclist historical sport information and the perfect cyclist sport information to generate a cycling sport training suggestion signal.

Moreover, in an embodiment of the cycling sport training suggestion system of the present disclosure mentioned above, the sport real-time analysis module includes a time series neural network architecture. After the sport real-time analysis module receives the cyclist real-time sense information, the route feature information, the cyclist historical sport information and the perfect cyclist sport information, the time series neural network architecture is configured to integrate and analyze the cyclist real-time sense information, the route feature information, the cyclist historical sport information and the perfect cyclist sport information to generate the cycling sport training suggestion signal.

Moreover, in an embodiment of the cycling sport training suggestion system of the present disclosure mentioned above, the cycling sport training suggestion system further includes a prompt module electrically connected to the sport real-time analysis module. Moreover, after the cycling sport training suggestion signal is generated, the sport real-time analysis module is configured to transmit the cycling sport training suggestion signal to the prompt module. After the prompt module receives the cycling sport training suggestion signal, the prompt module is configured to reveal a content of the cycling sport training suggestion signal.

Moreover, in an embodiment of the cycling sport training suggestion system of the present disclosure mentioned above, after the sport real-time analysis module receives the cyclist real-time sense information, the sport real-time analysis module is configured to transmit the cyclist real-time sense information to the sport role data base, so that the sport role data base is configured to store the cyclist real-time sense information.

Moreover, in an embodiment of the cycling sport training suggestion system of the present disclosure mentioned above, after the cycling sport training suggestion signal is generated, the sport real-time analysis module is configured to transmit the cycling sport training suggestion signal to the sport role data base, so that the sport role data base is configured to store the cycling sport training suggestion signal.

Moreover, in an embodiment of the cycling sport training suggestion system of the present disclosure mentioned above, the cyclist real-time sense information includes a heart rate information and a speed-and-pedal-frequency information corresponding to a specific route location.

Moreover, in an embodiment of the cycling sport training suggestion system of the present disclosure mentioned above, the cycling sport training suggestion system is applied to a bicycle apparatus and a cyclist. The bicycle apparatus is electrically connected to the route feature module. The cyclist pedals the bicycle apparatus. The cycling sport training suggestion system further includes a heart rate sensor electrically connected to the cyclist information module. Moreover, when the route feature module transmits the route feature information to the sport real-time analysis module, the route feature module is configured to control a pedaling difficulty of the bicycle apparatus based on the route feature information. The heart rate sensor is configured to be worn by the cyclist to sense the cyclist to obtain the heart rate information. After the heart rate sensor obtains the heart rate information, the heart rate sensor is configured to transmit the heart rate information to the cyclist information module.

Moreover, in an embodiment of the cycling sport training suggestion system of the present disclosure mentioned above, the cycling sport training suggestion system further includes a speed-and-pedal-frequency sensor electrically connected to the cyclist information module and the bicycle apparatus. Moreover, the speed-and-pedal-frequency sensor is configured to sense a speed and a pedal frequency of the bicycle apparatus to obtain the speed-and-pedal-frequency information. After the speed-and-pedal-frequency sensor obtains the speed-and-pedal-frequency information, the speed-and-pedal-frequency sensor is configured to transmit the speed-and-pedal-frequency information to the cyclist information module.

Moreover, in an embodiment of the cycling sport training suggestion system of the present disclosure mentioned above, the prompt module is a display. After the display receives the cycling sport training suggestion signal, the display is configured to display the content of the cycling sport training suggestion signal.

Moreover, in an embodiment of the cycling sport training suggestion system of the present disclosure mentioned above, the prompt module is a speaker. After the speaker receives the cycling sport training suggestion signal, the speaker is configured to play the content of the cycling sport training suggestion signal.

The advantage of the present disclosure is to improve the cycling efficiency of the cyclist.

Please refer to the detailed descriptions and FIGURES of the present disclosure mentioned below for further understanding the technology, method and effect of the present disclosure achieving the predetermined purposes. It believes that the purposes, characteristic and features of the present disclosure can be understood deeply and specifically. However, the figures are only for references and descriptions, but the present disclosure is not limited by the FIGURES.

### BRIEF DESCRIPTION OF THE DRAWING

FIG. 1 shows a block diagram of an embodiment of the cycling sport training suggestion system of the present disclosure.

### DETAILED DESCRIPTION

In the present disclosure, numerous specific details are provided, to provide a thorough understanding of embodiments of the disclosure. Persons of ordinary skill in the art will recognize, however, that the present disclosure can be practiced without one or more of the specific details. In other instances, well-known details are not shown or described to avoid obscuring aspects of the present disclosure. Now please refer to the figures for the explanation of the technical content and the detailed description of the present disclosure:

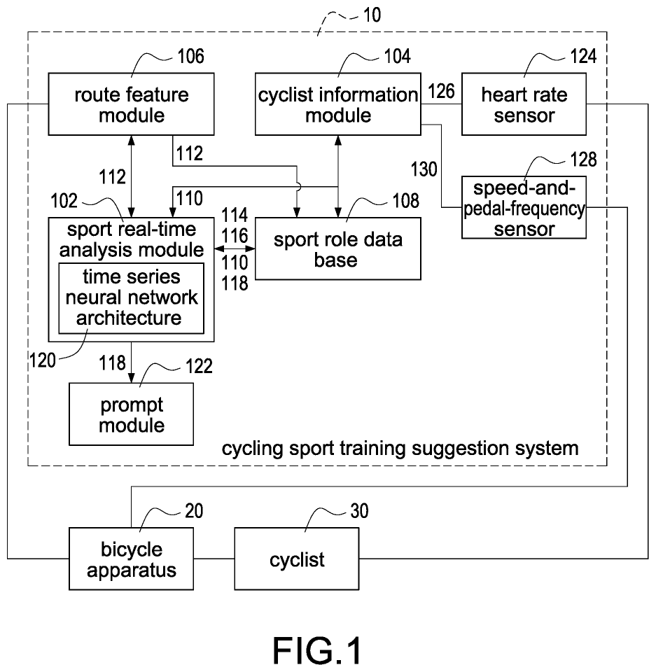

FIG. 1 shows a block diagram of an embodiment of the cycling sport training suggestion system 10 of the present disclosure. A cycling sport training suggestion system 10 of the present disclosure includes a sport real-time analysis module 102, a cyclist information module 104, a route feature module 106, a sport role data base 108, a prompt module 122, a heart rate sensor 124 and a speed-and-pedal-frequency sensor 128. The sport real-time analysis module 102 includes a time series neural network architecture 120. The components mentioned above are electrically connected to each other. The cycling sport training suggestion system 10 of the present disclosure is applied to a bicycle apparatus 20 and a cyclist 30. The bicycle apparatus 20 is electrically connected to the route feature module 106 and the speed-and-pedal-frequency sensor 128. The cyclist 30 pedals the bicycle apparatus 20 and wears the heart rate sensor 124.

First, the cyclist 30 rides on the bicycle apparatus 20, and inputs a personal information of the cyclist 30 on, for example, a touch screen (not shown in FIG. 1), and selects a route to practice riding (which may be a real route or a virtual route, and which corresponds to a route feature information 112), and presses a start button (not shown in FIG. 1).

Then, the route feature module 106 knows the route selected by the cyclist 30 to practice riding through the touch screen and transmits the route feature information 112 corresponding to the route to the sport real-time analysis module 102 and the sport role data base 108. The route feature module 106 controls a pedaling difficulty of the bicycle apparatus 20 based on the route feature information 112 (for example, when simulating a downhill road section, the pedaling difficulty is looser; when simulating an uphill road section, the pedaling difficulty is tighter). The sport role data base 108 knows the personal information of the cyclist 30 through the touch screen. After the sport role data base 108 receives the route feature information 112, the sport role data base 108 transmits a cyclist historical sport information 114 (belonging to the cyclist 30 and corresponding to the route feature information 112) and a perfect cyclist sport information 116 (corresponding to the route feature information 112) to the sport real-time analysis module 102.

Then, when the cyclist 30 pedals the bicycle apparatus 20, the heart rate sensor 124 senses the cyclist 30 to obtain a heart rate information 126 and transmits the heart rate information 126 to the cyclist information module 104. The speed-and-pedal-frequency sensor 128 senses a speed and a pedal frequency of the bicycle apparatus 20 to obtain a speed-and-pedal-frequency information 130 and transmits the speed-and-pedal-frequency information 130 to the cyclist information module 104. After the cyclist information module 104 receives the heart rate information 126 and the speed-and-pedal-frequency information 130, the cyclist information module 104 integrates the heart rate information 126 and the speed-and-pedal-frequency information 130 to obtain a cyclist real-time sense information 110. Namely, the cyclist real-time sense information 110 includes the heart rate information 126 and the speed-and-pedal-frequency information 130 corresponding to a specific route location. After the cyclist information module 104 obtains the cyclist real-time sense information 110, the cyclist information module 104 transmits the cyclist real-time sense information 110 to the sport real-time analysis module 102.

After the sport real-time analysis module 102 receives the cyclist real-time sense information 110, the route feature information 112, the cyclist historical sport information 114 and the perfect cyclist sport information 116, the time series neural network architecture 120 of the sport real-time analysis module 102 is configured to integrate and analyze the cyclist real-time sense information 110, the route feature information 112, the cyclist historical sport information 114 and the perfect cyclist sport information 116 to generate a cycling sport training suggestion signal 118.

After the cycling sport training suggestion signal 118 is generated, the sport real-time analysis module 102 transmits the cycling sport training suggestion signal 118 to the prompt module 122 and the sport role data base 108. After the prompt module 122 receives the cycling sport training suggestion signal 118, the prompt module 122 is configured to reveal a content of the cycling sport training suggestion signal 118. The prompt module 122 may be a display. After the display receives the cycling sport training suggestion signal 118, the display displays the content of the cycling sport training suggestion signal 118 for the cyclist 30 to watch. Or the prompt module 122 may be a speaker. After the speaker receives the cycling sport training suggestion signal 118, the speaker plays the content of the cycling sport training suggestion signal 118 for the cyclist 30 to listen to. The content may be, for example: “Currently it is a downhill road section; please slow down your pedaling speed”, “Currently it is an uphill road section, please increase your pedaling speed”, and so on.

After the sport real-time analysis module 102 receives the cyclist real-time sense information 110, the sport real-time analysis module 102 also transmits the cyclist real-time sense information 110 to the sport role data base 108. After the sport role data base 108 receives the cyclist real-time sense information 110 and the cycling sport training suggestion signal 118, the sport role data base 108 stores the cyclist real-time sense information 110 and the cycling sport training suggestion signal 118 as the cyclist historical sport information 114 (belonging to the cyclist 30 and corresponding to the route feature information 112) for the next time.

In an embodiment of the present disclosure but not limiting the present disclosure, the sport real-time analysis module 102 (including the time series neural network architecture 120), the cyclist information module 104, the route feature module 106 and the sport role data base 108 are respective microprocessors, or may be integrated into a single microprocessor. The perfect cyclist sport information 116 is the information recorded by the best cyclist 30 (such as a bicycle race champion) riding the route, including the heart rate information 126, the speed-and-pedal-frequency information 130 and so on of the best cyclist 30.

The advantage of the present disclosure is to improve the cycling efficiency of the cyclist.

Although the present disclosure has been described with reference to the embodiment thereof, it will be understood that the disclosure is not limited to the details thereof. Various substitutions and modifications have been suggested in the foregoing description, and others will occur to those of ordinary skill in the art. Therefore, all such substitutions and modifications are intended to be embraced within the scope of the disclosure as defined in the appended claims.

What is claimed is:

1. A cycling sport training suggestion system (10) comprising:
a sport real-time analysis module (102);
a cyclist information module (104) electrically connected to the sport real-time analysis module (102);
a route feature module (106) electrically connected to the sport real-time analysis module (102); and
a sport role data base (108) electrically connected to the sport real-time analysis module (102), the cyclist information module (104) and the route feature module (106),
wherein the cyclist information module (104) is configured to transmit a cyclist real-time sense information (110) to the sport real-time analysis module (102); the route feature module (106) is configured to transmit a route feature information (112) to the sport real-time analysis module (102); the sport role data base (108) is configured to transmit a cyclist historical sport information (114) and a perfect cyclist sport information (116) to the sport real-time analysis module (102); after the sport real-time analysis module (102) receives the cyclist real-time sense information (110), the route feature information (112), the cyclist historical sport information (114) and the perfect cyclist sport information (116), the sport real-time analysis module (102) is configured to integrate and analyze the cyclist real-time sense information (110), the route feature information (112), the cyclist historical sport information (114) and the perfect cyclist sport information (116) to generate a cycling sport training suggestion signal (118).

2. The cycling sport training suggestion system (10) of claim 1, wherein the sport real-time analysis module (102) comprises a time series neural network architecture (120); after the sport real-time analysis module (102) receives the cyclist real-time sense information (110), the route feature information (112), the cyclist historical sport information (114) and the perfect cyclist sport information (116), the time series neural network architecture (120) is configured to integrate and analyze the cyclist real-time sense information (110), the route feature information (112), the cyclist historical sport information (114) and the perfect cyclist sport information (116) to generate the cycling sport training suggestion signal (118).

3. The cycling sport training suggestion system (10) of claim 2, further comprising:
a prompt module (122) electrically connected to the sport real-time analysis module (102),
wherein after the cycling sport training suggestion signal (118) is generated, the sport real-time analysis module (102) is configured to transmit the cycling sport training suggestion signal (118) to the prompt module (122); after the prompt module (122) receives the cycling sport training suggestion signal (118), the prompt module (122) is configured to reveal a content of the cycling sport training suggestion signal (118).

4. The cycling sport training suggestion system (10) of claim 3, wherein after the sport real-time analysis module (102) receives the cyclist real-time sense information (110), the sport real-time analysis module (102) is configured to transmit the cyclist real-time sense information (110) to the sport role data base (108), so that the sport role data base (108) is configured to store the cyclist real-time sense information (110).

5. The cycling sport training suggestion system (10) of claim 4, wherein after the cycling sport training suggestion signal (118) is generated, the sport real-time analysis module (102) is configured to transmit the cycling sport training suggestion signal (118) to the sport role data base (108), so that the sport role data base (108) is configured to store the cycling sport training suggestion signal (118).

6. The cycling sport training suggestion system (10) of claim 5, wherein the cyclist real-time sense information (110) comprises a heart rate information (126) and a speed-and-pedal-frequency information (130) corresponding to a specific route location.

7. The cycling sport training suggestion system (10) of claim 6, the cycling sport training suggestion system (10) applied to a bicycle apparatus (20) and a cyclist (30), the bicycle apparatus (20) electrically connected to the route feature module (106), the cyclist (30) pedaling the bicycle apparatus (20), the cycling sport training suggestion system (10) further comprising:
a heart rate sensor (124) electrically connected to the cyclist information module (104),
wherein when the route feature module (106) transmits the route feature information (112) to the sport real-time analysis module (102), the route feature module (106) is configured to control a pedaling difficulty of the bicycle apparatus (20) based on the route feature information (112); the heart rate sensor (124) is configured to be worn by the cyclist (30) to sense the cyclist (30) to obtain the heart rate information (126); after the heart rate sensor (124) obtains the heart rate information (126), the heart rate sensor (124) is configured to transmit the heart rate information (126) to the cyclist information module (104).

8. The cycling sport training suggestion system (10) of claim 7, further comprising:
a speed-and-pedal-frequency sensor (128) electrically connected to the cyclist information module (104) and the bicycle apparatus (20),
wherein the speed-and-pedal-frequency sensor (128) is configured to sense a speed and a pedal frequency of the bicycle apparatus (20) to obtain the speed-and-pedal-frequency information (130); after the speed-and-pedal-frequency sensor (128) obtains the speed-and-pedal-frequency information (130), the speed-and-pedal-frequency sensor (128) is configured to transmit the speed-and-pedal-frequency information (130) to the cyclist information module (104).

9. The cycling sport training suggestion system (10) of claim 8, wherein the prompt module (122) is a display; after the display receives the cycling sport training suggestion signal (118), the display is configured to display the content of the cycling sport training suggestion signal (118).

10. The cycling sport training suggestion system (10) of claim 8, wherein the prompt module (122) is a speaker; after the speaker receives the cycling sport training suggestion signal (118), the speaker is configured to play the content of the cycling sport training suggestion signal (118).
